(19) United States
(12) Patent Application Publication
(10) Pub. No.: US 2025/0139473 A1
(43) Pub. Date: May 1, 2025

(54) Method And Apparatus For Designing Experiment

(71) Applicant: SK bioscience Co., Ltd., Gyeonggi-do (KR)

(72) Inventors: Dae Keun KWON, Gyeonggi-do (KR); Geon Hyeong LEE, Gyeonggi-do (KR); Hae Bin SHIN, Gyeonggi-do (KR); Seung Hwan KONG, Gyeonggi-do (KR); Gi Eun KWAK, Gyeonggi-do (KR)

(73) Assignee: SK bioscience Co., Ltd., Gyeonggi-do (KR)

(21) Appl. No.: 18/932,238

(22) Filed: Oct. 30, 2024

(30) Foreign Application Priority Data
Oct. 31, 2023 (KR) 10-2023-0148452

(51) Int. Cl. G06N 5/045

(52) U.S. Cl. CPC G06N 5/045

(57) ABSTRACT

A method and apparatus for designing an experiment are disclosed. The method of designing an experiment includes generating a candidate value of a critical process parameter (CPP) based on a condition for the CPP corresponding to a target response, obtaining a prediction value of the target response for the candidate value of the CPP, based on a response prediction model trained to estimate a function of the target response for the CPP, and outputting an experimental condition set of the CPP based on the prediction value of the target response.

### CROSS-REFERENCE TO RELATED APPLICATION

This application claims the priority benefit of Korean Patent Application No. 10-2023-0148452, filed on Oct. 31, 2023, in the Korean Intellectual Property Office, the disclosure of which is incorporated herein by reference for all purposes.

### BACKGROUND

### 1. Field

One or more embodiments relate to a method and apparatus for designing an experiment.

### 2. Description of the Related Art

The designing of experiments, such as validation experiments for vaccine development, having a plurality of variables is a lengthy and complex process. For the efficiency of an experimental design process, automation technology is required for a process of selecting a critical process parameter (CPP), that is, an important variable having a high contribution in experimental results, and a process of deducing an optimal experimental design by setting a value of the CPP. Upon this requirement, technology has been developed to collect and analyze data automatically in an experimental process, estimate the CPP by using machine learning and artificial intelligence algorithms, and predict experimental results from the CPP value.

### SUMMARY

Aspects provide technology for obtaining experimental conditions for obtaining a target response by predicting experimental results according to the estimation of a critical process parameter (CPP) and the setting of a value of the CPP by using a training model.

However, technical aspects are not limited to the foregoing aspects, and there may be other technical aspects.

According to an aspect, there is provided a method of designing an experiment including generating a candidate value of a CPP based on a condition for the CPP corresponding to a target response; obtaining a prediction value of the target response for the candidate value of the CPP, based on a response prediction model trained to estimate a function of the target response for the CPP; and outputting an experimental condition set of the CPP based on the prediction value of the target response.

The method may further include training the response prediction model based on experimental data for measuring the target response for the CPP.

The method may further include determining at least some of process parameters of the CPP, based on a multivariate analysis model for evaluating the contribution of the process parameters for the target response.

The multivariate analysis model may include a model for evaluating the contribution of the process parameters for the target response by estimating a Shapley additive explanations (SHAP) value for the target response of the process parameters.

The method may further include determining a range of a value of the CPP, based on experimental data for measuring the target response from at least some of the process parameters.

The method may further include setting a range selected by an input of a user within a range of the determined range of the value of the CPP as the condition for the CPP.

The outputting of the experimental condition set of the CPP may include filtering the candidate value of the CPP to be included in the experimental condition set, based on a condition for the target response.

The generating of the candidate value of the CPP may include determining a random number satisfying the condition for the CPP as the candidate value of the CPP.

The generating of the candidate value of the CPP may include obtaining the candidate value of the CPP from a language model, based on a prompt corresponding to a condition for the target response and experimental data for measuring the target response for the CPP.

The generating of the candidate value of the CPP from the language model may include obtaining embedding data of the experimental data for measuring the target response for the CPP; and obtaining the candidate value of the CPP from the language model, based on the prompt corresponding to the condition for the target response and the embedding data of the experimental data.

According to another aspect, there is provided a method of designing an experiment including generating a candidate value of a CPP based on a condition for the CPP; obtaining a prediction value of a first target response for the candidate value of the CPP; filtering the candidate value of the CPP based on a condition for the first target response and the prediction value of the first target response; obtaining a prediction value of a second target response for the filtered candidate value of the CPP; and outputting an experimental condition set of the CPP by filtering the candidate value of the CPP based on a condition for the second target response and the prediction value of the second target response.

The obtaining of the prediction value of the first target response may include obtaining the prediction value of the first target response for the candidate value of the CPP, based on a response prediction model trained to estimate a function of the first target response for the CPP.

The obtaining of the prediction value of the second target response may include obtaining the prediction value of the second target response for the candidate value of the CPP, based on a response prediction model trained to estimate a function of the second target response for the CPP.

According to another aspect, there is provided an apparatus including a processor configured to generate a candidate value of a CPP based on a condition for the CPP corresponding to a target response, obtain a prediction value of the target response for the candidate value of the CPP, based on a response prediction model trained to estimate a function of the target response for the CPP, and output an experimental condition set of the CPP based on the prediction value of the target response.

The processor may train the response prediction model based on experimental data for measuring the target response for the CPP.

The processor may determine at least some of process parameters as the CPP, based on a multivariate analysis model for evaluating the contribution of the process parameters for the target response, in which the multivariate analysis model may include a model for evaluating the contribution of the process parameters for the target response by estimating a SHAP value for the target response of the process parameters.

The processor may determine a range of a value of the CPP, based on experimental data for measuring the target response from at least some of the process parameters.

According to another aspect, there is provided an apparatus including a processor configured to generate a candidate value of a CPP based on a condition for the CPP, obtain a prediction value of a first target response for the candidate value of the CPP, filter the candidate value of the CPP based on a condition for the first target response and the prediction value of the first target response, obtain a prediction value of a second target response for the filtered candidate value of the CPP, and output an experimental condition set of the CPP by filtering the candidate value of the CPP based on a condition for the second target response and the prediction value of the second target response.

The processor, when obtaining the prediction value of the first target response, may obtain the prediction value of the first target response for the candidate value of the CPP, based on a response prediction model trained to estimate a function of the first target response for the CPP, and, when obtaining the prediction value of the second target response, may obtain the prediction value of the second target response for the candidate value of the CPP, based on the response prediction model trained to estimate a function of the second target response for the CPP.

### BRIEF DESCRIPTION OF THE DRAWINGS

FIG. 1 is a flowchart illustrating an operation of a method of designing an experiment, according to an embodiment.

FIG. 2 is a diagram illustrating a process of a method of designing an experiment for obtaining an experimental condition set of a critical process parameter (CPP), according to an embodiment.

FIG. 3 is a diagram illustrating an operation of obtaining a candidate value of a CPP by using a language model, according to an embodiment.

FIG. 4 is a diagram illustrating an input and output of a language model, according to an embodiment.

FIG. 5 is a flowchart illustrating an operation of a method of designing an experiment, according to an embodiment.

FIG. 6 is a diagram illustrating a process of a method of designing an experiment for obtaining an experimental condition set of a CPP by filtering a candidate value of the CPP under a condition for a plurality of target responses, according to an embodiment.

FIGS. 7A and 7B are diagrams each illustrating an interface for providing a method of designing an experiment, according to an embodiment.

FIG. 8 is a diagram illustrating a configuration of an apparatus according to an embodiment.

FIG. 9 is a chart illustrating a degree of impact of a process parameter on the determining of a target response, according to an embodiment.

FIG. 10 is a graph illustrating the importance of a process parameter to a target response by experiment, according to an embodiment.

### DETAILED DESCRIPTION

The following detailed structural or functional description is provided as an example only and various alterations and modifications may be made to embodiments. Here, examples are not construed as limited to the disclosure and should be understood to include all changes, equivalents, and replacements within the idea and the technical scope of the disclosure.

Terms, such as first, second, and the like, may be used herein to describe various components. Each of these terminologies is not used to define an essence, order or sequence of a corresponding component but used merely to distinguish the corresponding component from other component(s). For example, a first component may be referred to as a second component, and similarly the second component may also be referred to as the first component.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/including” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like elements and a repeated description related thereto will be omitted.

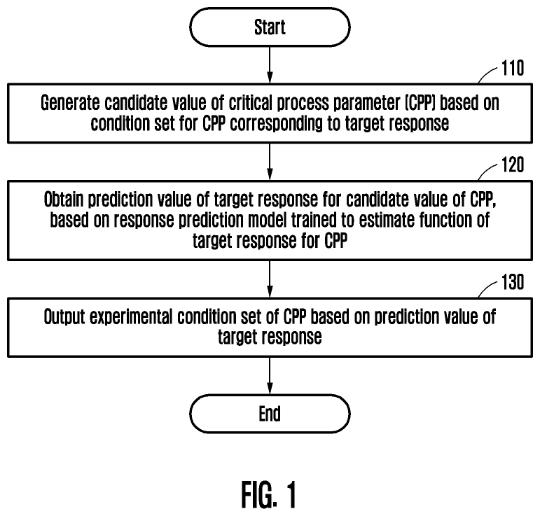

FIG. 1 is a flowchart illustrating an operation of a method of designing an experiment, according to an embodiment.

The experimental design method, according to an embodiment, may be performed by a processor of an apparatus for designing an experiment. The apparatus is an electronic device including at least one processor and may include, for example, at least one of a server and a user terminal (e.g., a personal computer (PC), a smartphone, a tablet, a wearable device, etc.). The hardware configuration of the apparatus is described in detail below.

Referring to FIG. 1, the experimental design method, according to an embodiment, may include operation 110 of generating a candidate value of a critical process parameter (CPP) based on a condition set for the CPP corresponding to a target response. The CPP may include one or more process parameters selected from among process parameters. The process parameters are factors, which are the variables of an experiment, that may include, for example, a molecular weight, oxygen saturation, dioxide saturation, humidity, temperature, stirring speed, dioxide concentration, oxygen supply pressure, viscous medium injection speed, pDNA sequence length, or a ratio of sugar to protein. More specifically, the process parameters for typhoid vaccine production may include, for example, a sugar-protein reaction ratio, a PS (Polysaccharide) scale (Polysaccharide consumption), Mw (molecular weight), reaction temperature, reaction time, stirring speed, pH, a molar equivalent of an oxidizing agent, or a molar equivalent of a conjugating reagent. The target response is an element that is the objective of an experiment and may include, for example, at least one of yield and purity.

According to an embodiment, the experimental design method may include an operation of determining at least some of process parameters of the CPP, based on a multivariate analysis model for evaluating the contribution of the process parameters for the target response. The multivariate analysis model may be a model for analyzing the effects of a plurality of variables. The variables of the multivariate analysis model for evaluating the contribution of the process parameters for the target response may correspond to the process parameters, and the effects of the variables may correspond to the contribution of the process parameters for the target response. The multivariate analysis model may include a training model and may include, for example, a model trained to estimate a Shapley additive explanations (SHAP) value. For example, the multivariate analysis model may include various learning-based models for estimating feature importance, besides the estimating of a SHAP value. A learning-based multivariate analysis model may be trained based on actual experimental data including the data of the target response by the process parameters. For example, the multivariate analysis model may include a model for evaluating the contribution of the process parameters for the target response by estimating a SHAP value for the target response of the process parameters.

Based on experimental data corresponding to the training data of the multivariate analysis model, a training algorithm may be determined for the training of the multivariate analysis model. In other words, prediction modeling may be applied before generating an experimental condition set. In this case, the training algorithm may include random forest, gradient boosted trees, ridge (L2) regression, lasso (L1) regression, light gradient boosting machine (GBM), XGBoost, or decision trees, and, preferably, may be random forest and XGBoost, but examples are not limited thereto.

The evaluation metric of the training algorithm may include a root mean squared error (RMSE), a mean absolute error (MAE), a mean squared error (MSE), a mean absolute percentage error (MAPE), or a root mean squared log error (RMSLE), and the MAPE may be used for the evaluation of the training algorithm, but examples are not limited thereto.

The MAPE is an evaluation metric that is used widely to verify whether a regression model is trained well and is similar to the MAE, but the major difference from the MAE is that the MAPE is a probability value derived by dividing an actual correct answer value. The equation of the MAPE is shown in Equation 1 below.

$$M A P E = \frac{100}{n} \times {\sum }_{i = 1}^{n} \vert \frac{{Y}_{i} − {\hat{Y}}_{i}}{{Y}_{i}} \vert \qquad [ Equation l ]$$

In Equation 1, Yᵢ denotes the actual correct answer value, and denotes a prediction value. The MAPE has a probability value between 0 to 100%, and thus, the results may be readily interpreted. The MAPE has a value related to a ratio that is not related to the size of a data value, and thus, the performance comparison of data may be readily performed.

The performance of the training algorithm may be evaluated for each piece of experimental data for the training of the multivariate analysis model by using the evaluation metric. The training algorithm may be determined for each piece of experimental data according to the evaluation results.

For example, the multivariate analysis model may be trained through typhoid serotype experimental data by using various training algorithms. When measuring an error rate by each training algorithm by using the MAPE evaluation metric after training the multivariate analysis model, XGBoost had a low error rate of 18.8%. Thus, XGBoost was selected as a training algorithm appropriate for the experimental data.

However, the appropriate training algorithm may vary depending on experimental data. The applying of the algorithm as the optimal algorithm for predicting the typhoid serotype experimental data was identified as XGBoost is just an example. The XGBoost algorithm may not be the most appropriate for all experimental data. Even for the same typhoid serotype experimental data, the training algorithm may vary depending on the content of the data. Thus, it should not be interpreted that examples are limited to the training algorithm.

A process parameter having high contribution evaluated by the multivariate analysis model may be determined as the CPP. For example, a process parameter having contribution greater than or equal to a threshold value or top n (here, n is a random natural number) process parameters or top m % (here, m is a random positive real number) of process parameters in terms of contribution may be determined as the CPP.

The condition for the CPP may be a condition for a value of the CPP to be used for an experiment. For example, the condition for the CPP may include a condition for determining the value of the CPP, such as a value of each CPP and a range of the value of each CPP, to be used for the experiment.

The CPP may be a molecular weight (Mw), a conjugated protein-polysaccharide reaction ratio (reaction ration P:S fraction), a reaction concentration, a reaction scale, a polysaccharide reaction scale, a reaction time, a reaction temperature, a conjugated reaction reagent concentration, a conjugation capping reagent concentration, a yield, a pure yield, a ratio of polysaccharide to conjugated protein (S/P ratio), total protein, total polysaccharide, pure saccharide, a purity ratio, free saccharide, MSD, MAALS, or the like. The CPP may vary depending on input data.

The molecular weight may be total mass had by a substance molecule, such as polysaccharide or polysaccharide-protein conjugate. The reaction ratio may be a content-based ratio of conjugated protein to polysaccharide used for conjugation reaction. The reaction concentration may be the concentration of polysaccharide used for conjugation reaction. The reaction scale may be the total content of polysaccharide used for conjugation reaction. The reaction time may be a time elapsing from the injection of a substance (e.g., activating polysaccharide, conjugated protein, or conjugated reagent in conjugation reaction) that is a reaction target until the capping. Free sugar (free saccharide) may be polysaccharide not participating in conjugation reaction and not formed of a polysaccharide-protein conjugate. A molecular size distribution (MSD) is a molecular weight distribution, may be a distribution of molecular weight values of sample substances, and, specifically in conjugation reaction, may be an indicator for confirming how uniform molecular weights had by substances formed of a conjugate are. A multi-angle laser scatter (MALS) may be a method of measuring a molecular weight or a molecular weight measured in the method.

The multivariate analysis model may be used for selecting a process parameter for vaccine production having a high yield. Specifically, the multivariate analysis model may be used for predicting a process parameter for generating a immunogenic composition at a high yield without many experiments. Specifically, the type of vaccines including immunogenic compositions may be tuberculosis, MMR, Japanese encephalitis, varicella, rotavirus, herpes zoster, yellow fever, influenza, hepatitis type B, diphtheria, tetanus, pertussis, polio, IPV, hemorrhagic influenza type B, hepatitis type A, pneumococcal, HPV, typhoid, nephrotic syndrome hemorrhagic fever, meningococcal, or RSV.

A Boruta algorithm is an example of a feature selection algorithm and includes an operation of validating a variable is a critical variable by using a binomial distribution. Thus, this algorithm has an advantageous effect that the importance of variables may be obtained together with statistical significance. For example, when training the multivariate analysis model with an input of streptococcal serotype-related training data by using Boruta SHAP obtained by applying SHAP, or an explainable artificial intelligence framework, to the Boruta algorithm, a total of 5 CPPs, which are confirmed as a reaction ratio (P:S) of 31%, a reaction concentration of 11%, a PS scale of 11%, and an Mw of 6% in order, were derived. It was confirmed that a reaction ratio (P:S) is the CPP corresponding to a yield fluctuation.

According to an embodiment, the experimental design method may include an operation of determining a range of a value of the CPP, based on experimental data measuring a target response from at least some of the CPP. For example, a range including values of actual CPPs including actual experimental data used for the training of the multivariate analysis model for determining the CPP may be determined as a range of a value of the CPP.

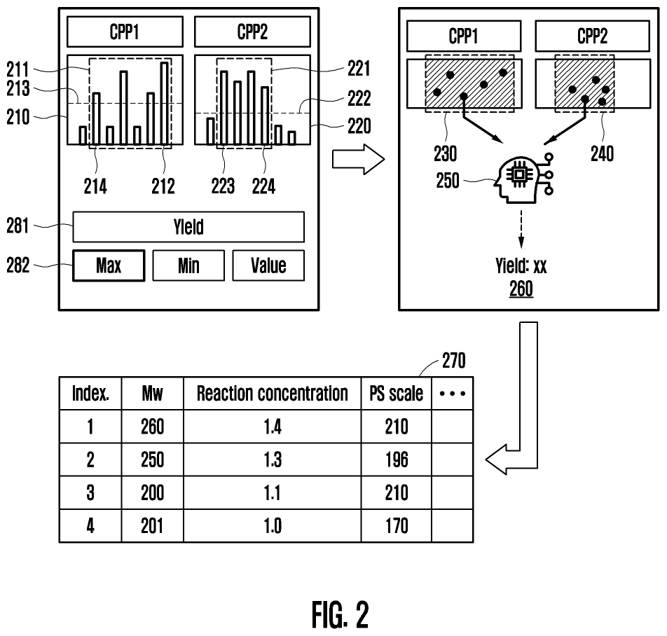

For example, referring to FIG. 2, a range 211 of a value of a first CPP (CPP1) and a range 221 of a value of a second CPP (CPP2) may be determined. The experimental frequency of a value of the first CPP may be obtained from the actual experimental data used for the training of the multivariate analysis model. Graph 210 corresponding to the first CPP included in FIG. 2 may be a graph having a horizontal axis as the value of the first CPP and a vertical axis as experimental frequency. The horizontal axis of graph 210 may indicate the value of the first CPP that increases gradually to the right. The vertical axis of graph 210 may indicate experimental frequency that increases gradually to the top. Referring to graph 210, the height of a stick corresponding to a first value 212 is the highest, which may refer that the number of experiments with the value of the first CPP as the first value 212 according to actual experimental data. Graph 220 corresponding to the second CPP may be a graph having a horizontal axis as the value of the second CPP and a vertical axis as experimental frequency.

For example, the range 211 of the value of the first CPP may be a range having a smallest value 214 of the first CPP as the lower limit and having the greatest value, that is, the first value 212, of the first CPP as the upper limit of the value of the first CPP having the experimental frequency greater than or equal to a first threshold value 213. Likewise, the range 221 of the value of the second CPP may be a range having a smallest value 223 of the second CPP as the lower limit and having a greatest value 224 of the second CPP as the upper limit of the value of the second CPP having the experimental frequency greater than or equal to a second threshold value 222. The first threshold value 213 for determining the range of the first CPP and the second threshold value 222 for determining the range of the second CPP may be determined independently of each other and may be the same as each other or different from each other.

According to an embodiment, the experimental design method may include an operation of setting a range selected by an input of a user within a range of the determined range of the value of the CPP as the condition for the CPP. The determined range of the value of the CPP may be provided to the user's terminal through a user interface. For example, the user may select at least some range of the range 211 of the value of the first CPP and/or the range 221 of the value of the second CPP or a value within the range. The selected range of the value of the CPP by the user may be set as the condition for the CPP. The user interface for setting the condition for the CPP is described in detail below.

Specifically, the parameters by each CPP may be as shown below.

The molecular weight (Mw) may be within a range of 0 to 500,000, 1 to 250,000, 10 to 100,000, 50 to 10,000, or 100 to 1,000 kDa. The polysaccharide reaction ratio (reaction ration P:S fraction) may be within a range of 0 to 100, 0.1 to 10, or 0.2 to 5. The reaction concentration may be within a range of 0 to 100, 0.1 to 10, or 0.2 to 5 mg/ml. The polysaccharide reaction scale may be within a range of 0 to 10,000, 1 to 5,000, 10 to 1,000, or 50 to 750. The reaction time may be within a range of 0 to 300, 1 to 150, 1 to 100, or 5 to 50 hr. The reaction temperature may be within a range of 0 to 100, 1 to 80, 2 to 50, or 10 to 40° C. The reaction reagent concentration may be within 0 to 100, 0.0001 to 50, 0.001 to 25, or 0.01 to 5. The reaction conjugation capping reagent concentration may be within 0 to 100, 0.001 to 50, 0.01 to 25, or 0.1 to 5. The yield may be within a range of 0 to 100, 0.1 to 99.9, or 1 to 90%, preferably, in a range greater than or equal to 40, 50, 60, 70, 80, or 90. The yield purity may be within a range of 0 to 100, 0.1 to 99.9, or 1 to 90, preferably, in a range greater than or equal to 40, 50, 60, 70, 80, or 90. The ratio of polysaccharide to S (S/P ratio) may be within a range of 0 to 100, 0.0001 to 50, 0.001 to 25, or 0.01 to 12. The total protein or the total polysaccharide may be within a range of 0 to 100,000, 1 to 10,000, 10 to 5,000, or 50 to 3,000. The pure saccharide may be within a range of 0 to 100,000, 1 to 10,000, 10 to 5,000, or 40 to 2,000. The purity ratio may be within a range of 0 to 100, 0.001 to 50, 0.01 to 25, or 0.1 to 5. The free saccharide may be within a free saccharide/% range of 0 to 100, 0 to 99.9, or 0 to 90, preferably, in a range greater than or equal to 40, 50, 60, 70, 80, or 90. The MSD may be within a molecular weight distribution/% range of 0 to 100, 0.1 to 99, or 30 to 99. The MALS may be within a range of 0 to 1,000,000, 1 to 500,000, 10 to 100,000, or 100 to 90,000 kDa.

Referring to FIG. 1 again, according to an embodiment, operation 110 of generating the candidate value of the CPP may include an operation of determining a random number satisfying the condition for the CPP as the candidate value of the CPP. For example, a random number may be generated within the determined range of the value of the CPP, and the generated random number may be determined as the candidate value of the CPP. For example, a plurality of random numbers may be generated, and the candidate value of the CPP may be determined. For example, the distribution of CPPs may be estimated within the determined range of the value of the CPP, and a random number may be generated based on the estimated distribution of CPPs.

According to an embodiment, operation 110 of generating the candidate value of the CPP may include an operation of obtaining the candidate value of the CPP from a language model, based on a prompt corresponding to a condition for the target response and experimental data for measuring the target response for the CPP. For example, an operation of generating the candidate value of the CPP from the language model may include an operation of obtaining embedding data of the experimental data for measuring the target response for the CPP and an operation of obtaining the candidate value of the CPP from the language model, based on the prompt corresponding to the condition for the target response and the embedding data of the experimental data. The method of obtaining the candidate value of the CPP from the language model is described in detail below.

According to an embodiment, the experimental design method may include operation 120 of obtaining a prediction value of the target response for the candidate value of the CPP, based on a response prediction model trained to estimate a function of the target response for the CPP.

According to an embodiment, the method may include an operation of training the response prediction model based on experimental data for measuring the target response for the CPP. The response prediction model may include a neural network trained based on experimental data for measuring the target response for the CPP. The trained response prediction model may output the prediction value of the target response when the value of the CPP is input.

For example, referring to FIG. 2, a candidate value 230 of the first CPP (CPP1) and a candidate value 240 of the second CPP (CPP2) may be input to a trained response prediction model 250. The response prediction model 250 may output a prediction value 260 of the target response (e.g., yield) corresponding to the candidate value 230 of the first CPP and the candidate value 240 of the second CPP. The prediction value 260 of the target response corresponding to all combinations of the candidate value 230 of the first CPP and the candidate value 240 of the second CPP may be obtained from the response prediction model 250.

Referring to FIG. 1 again, the experimental design method, according to an embodiment, may include operation 130 of outputting an experimental condition set of the CPP based on the prediction value of the target response. The experimental condition set may be a set of values of CPPs. For example, if the CPPs include the first CPP and the second CPP, the experimental condition set may include one or more sets of the value of the first CPP and the value of the second CPP. For example, referring to FIG. 2, each row of an experimental condition set 270 may indicate a set of values of the CPPs (e.g., molecular weight (Mw), reaction concentration, reaction scale, etc.).

Referring to FIG. 1 again, operation 130 of outputting of the experimental condition set of the CPP, according to an embodiment, may include an operation of filtering the candidate value of the CPP to be included in the experimental condition set, based on a condition for the target response. The condition for the target response is a condition set for the target response corresponding to the objective of an experiment, and, for example, may include at least one of a condition for maximizing the target response, a condition for minimizing the target response, a condition for obtaining the target response of a value belonging to a certain range, and a condition for obtaining the target response of a certain value.

The operation of filtering the candidate value of the CPP to be included in the experimental condition set may include an operation of selecting the candidate value of the CPP corresponding to the condition for the target response.

For example, if the condition for the target response is the condition for maximizing the target response, top n (here, n is a random natural number) candidate values of the CPPs having the highest prediction value of the target response, the candidate value of the CPP in top m % (here, m is a random positive real number) having the highest prediction value of the target response, or the candidate value of the CPP having the prediction value of the target response greater than or equal to a threshold value may be selected as the candidate value of the CPP to be included in the experimental condition set.

For example, if the condition for the target response is the condition for minimizing the target response, bottom n (here, n is a random natural number) candidate values of the CPPs having the highest prediction value of the target response, the candidate value of the CPP in the bottom m % (here, m is a random positive real number) having the highest prediction value of the target response, or the candidate value of the CPP having the prediction value of the target response less than or equal to the threshold value may be selected as the candidate value of the CPP to be included in the experimental condition set.

For example, if the condition for the target response is the condition for obtaining the target response of a value belonging to a certain range, the candidate value of the CPP with the prediction value of the target response belonging to the certain range or the candidate value of the CPP having a difference between the prediction value of the target response and the upper value or the lower value of the certain range being less than or equal to the threshold value may be selected as the candidate value of the CPP to be included in the experimental condition set.

For example, if the condition for the target response is the condition for obtaining the target response of a certain value, top n (here, n is a random natural number) candidate values of the CPPs in which the prediction value of the target response is the closest to the certain value, the candidate value of the CPP in top m % (here, m is a random positive real number) in which the prediction value of the target response is the closest to the certain value, or the candidate value of the CPP having a difference between the prediction value of the target response and the certain value being less than or equal to the threshold value may be selected as the candidate value of the CPP to be included in the experimental condition set.

For example, referring to FIG. 2, a target response 281 may be set to a yield, and a condition 282 for the target response may be set to the condition for maximizing the yield. The prediction value 260 of the target response corresponding to all combinations of the candidate value 230 of the first CPP and the candidate value 240 of the second CPP may be obtained from the response prediction model 250. Based on the condition 282 for the target response that maximizes the yield, the candidate value of the CPP to be included in the experimental condition set 270 may be selected from the obtained prediction value 260 of the target response. For example, top n (here, n is a random natural number) (e.g., 4) candidate values of the CPP having the highest prediction value 260 of the target response may be selected and output as the experimental condition set 270.

Referring to FIG. 1 again, the experimental condition set of the CPP output in operation 130 may be a combination of values of CPPs having a high probability of obtaining experimental results corresponding to the condition for the target response. With obtaining the experimental condition set of the CPP by using the response prediction model, the number of experiments may be reduced by narrowing a range of a value to be an experimental target of the CPP.

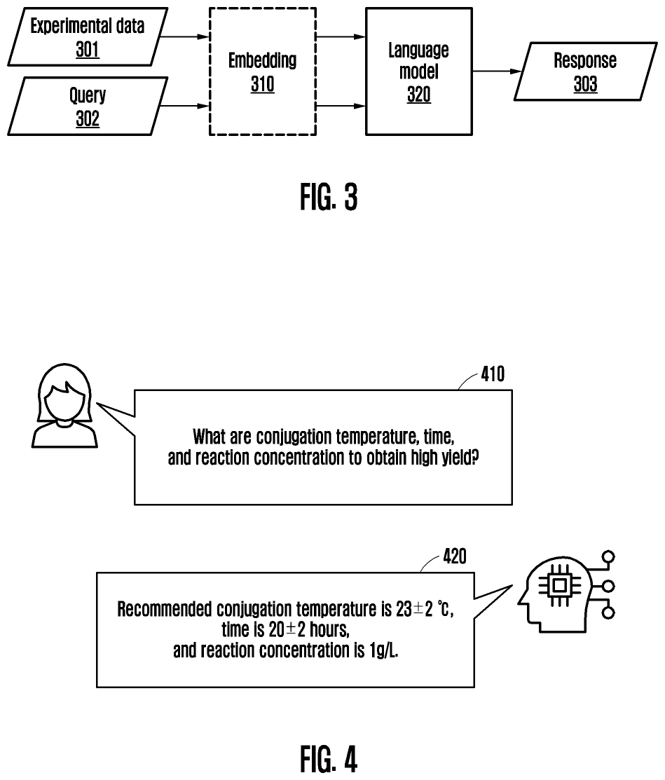

FIG. 3 is a diagram illustrating an operation of obtaining a candidate value of a CPP by using a language model, according to an embodiment.

Referring to FIG. 3, the candidate value of the CPP may be obtained based on a response 303 of a language model 320. To obtain the candidate value of the CPP as the response 303, a prompt corresponding to a query 302 and experimental data 301 for measuring a target response for the CPP may be input to the language model 320.

The query 302 may include an input requesting a value of the CPP corresponding to a condition for the target response. For example, if the target response is a yield, and the condition for the target response is a condition for maximizing the yield, the query 302 may include an input requesting the value of the CPP for maximizing the yield.

The experimental data 301 may include a value of the target response measured as an experimental result from at least some of the values of the CPPs. The experimental data 301 may include the values of the CPPs used for an actual experiment and the value of the target response obtained by performing an experiment from the values of the CPPs. The experimental data 301 may include one or more data sets including a combination of the values of the CPPs and the value of the target response. The experimental data 301 may be processed in a predetermined form. For example, the experimental data 301 recorded in a document file may be processed as a table including each value by being indexed by the name and identification information of the CPP or the name or identification information of the target response.

The prompt may include an instruction requesting a response to the query 302 with reference to the experimental data 301. The prompt may be generated based on the embedding data of the query 302 and/or the experimental data 301.

According to an embodiment, embedding 310 may be performed on the experimental data 301 to be input to the language model 320. The embedding data obtained as a result of the embedding 310 of the experimental data 301 may be input to the language model 320. According to an embodiment, embedding 310 may be performed on the query 302 to be input to the language model 320. The embedding data obtained as a result of the embedding 310 of the query 302 may be input to the language model 320.

The language model 320 may output the response 303 corresponding to the input including the experimental data 301 and the query 302. The language model 320 may generate the response 303 to the query 302 with reference to the experimental data 301. The response 303 may include the set(s) of candidate values of CPPs corresponding to the condition for the target response.

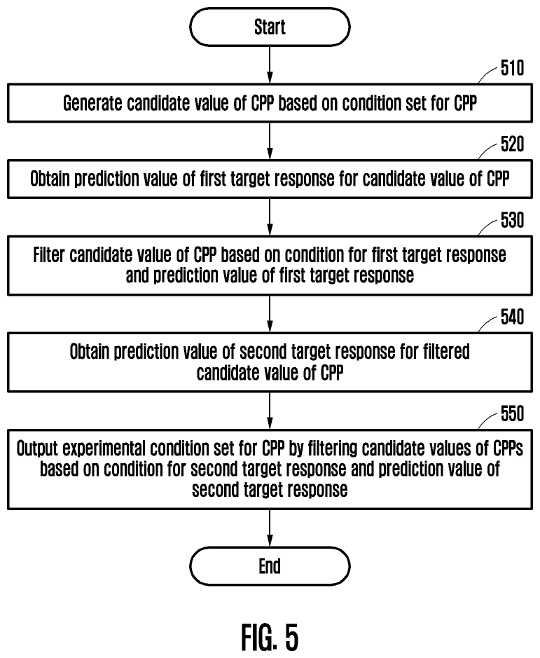

For example, referring to FIG. 4, a query 410 requesting a value of a CPP (e.g., conjugation temperature, time, and reaction concentration) to obtain a high target response (e.g., yield) may be input to a language model. In this case, together with the query 410, actual experimental data of the target response (e.g., yield) for the CPP (e.g., at least one of the conjugation temperature, time, and reaction concentration) may be input to the language model. The language model may output a response 420 including a candidate value of the CPP (e.g., the conjugation temperature, time, and reaction concentration) to maximize the target response (e.g., yield) in response to the input query 410 and experimental data.

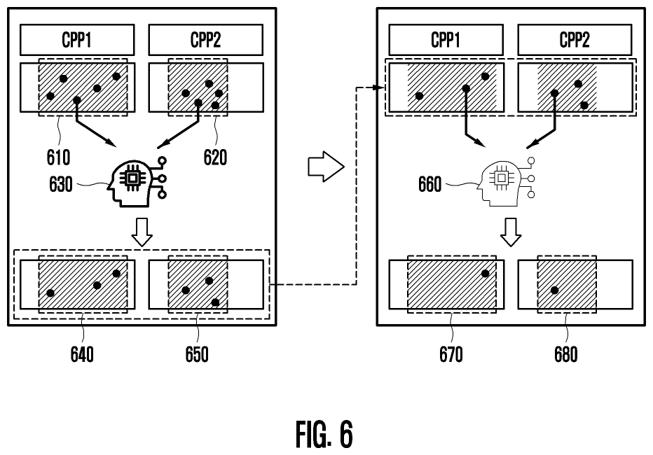

FIG. 5 is a flowchart illustrating an operation of a method of designing an experiment, according to an embodiment.

The experimental design method, according to an embodiment, may be performed by a processor of an apparatus for designing an experiment. The apparatus is an electronic device including at least one processor and may include, for example, at least one of a server and a user terminal (e.g., a PC, a smartphone, a tablet, a wearable device, etc.). The hardware configuration of the apparatus is described in detail below.

Referring to FIG. 5, the experimental design method, according to an embodiment, may include operation 510 of generating a candidate value of a CPP based on a condition set for the CPP. Operation 510 may correspond to operation 110 described above with reference to FIG. 1.

According to an embodiment, the experimental design method may include operation 520 of obtaining a prediction value of a first target response for a candidate value of the CPP. The first target response may be one target response of a target responses set corresponding to the CPP. The target responses set corresponding to the CPP may include two or more target responses.

For example, operation 520 of obtaining the prediction value of the first target response may include obtaining the prediction value of the first target response for the candidate value of the CPP, based on a response prediction model trained to estimate a function of the first target response for the CPP. Hereinafter, the response prediction model trained to estimate a function of the first target response may be referred to as a first response prediction model. The first response prediction model may be included in the response prediction model described above with reference to FIG. 1. The first response prediction model may include a neural network trained based on experimental data for measuring the first target response for the CPP. The trained first response prediction model may output the prediction value of the first target response when the value of the CPP is input.

The experimental design method, according to an embodiment, may include operation 530 of filtering a candidate value of the CPP based on a condition for the first target response and the prediction value of the first target response. The condition for the first target response is a condition set for the first target response corresponding to the objective of an experiment, and, for example, may include at least one of a condition for maximizing the first target response, a condition for minimizing the first target response, a condition for obtaining the first target response of a value belonging to a certain range, and a condition for obtaining the first target response of a certain value. Based on the condition for the first target response and the prediction value of the first target response, the candidate value of the CPP corresponding to the condition for the first target response may be selected. For example, operation 530 may correspond to the operation of filtering the candidate value of the CPP to be included in an experimental condition set, based on the condition for the target response, described above with reference to FIG. 1.

According to an embodiment, the experimental design method may include operation 540 of obtaining a prediction value of a second target response for the filtered candidate value of the CPP. The second target response may be one target response of a target responses set corresponding to the CPP. The second target response may be a target response that is different from the first target response.

For example, operation 540 of obtaining the prediction value of the second target response may include obtaining the prediction value of the second target response for the candidate value of the CPP, based on a response prediction model trained to estimate a function of the second target response for the CPP. Hereinafter, the response prediction model trained to estimate a function of the second target response may be referred to as a second response prediction model. The second response prediction model may be included in the response prediction model described above with reference to FIG. 1. The second response prediction model may include a neural network trained based on experimental data for measuring the second target response for the CPP. The trained second response prediction model may output the prediction value of the second target response when the value of the CPP is input.

The filtered CPP may include the candidate value of the CPP selected based on the condition for the first target response in operation 530. Instead of obtaining the prediction value of the second target response for all the candidate values of the CPPs obtained in operation 510, the prediction value of the second target response for the candidate value of the CPP selected based on the condition for the first target response may be obtained.

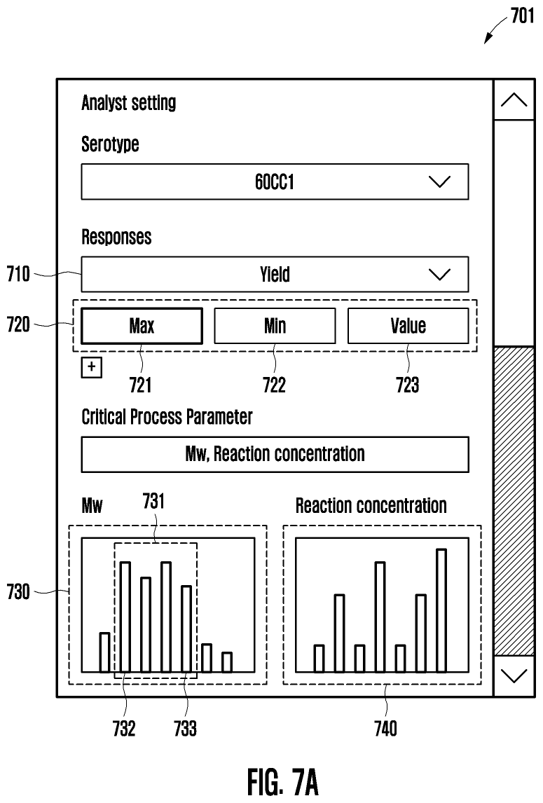

For example, referring to FIG. 6, the prediction value of the first target response may be obtained from a first response prediction model 630 for all the combinations of candidate values 610 of a first CPP and candidate values 620 of a second CPP obtained in operation 510. The candidate values 610 of the first CPP and the candidate values 620 of the second CPP may be filtered based on the condition for the first target response and the prediction value of the first target response. The prediction value of the second target response may be obtained from a second response prediction model 660 for all the combinations of filtered candidate values 640 of the first CPP and filtered candidate values 650 of the second CPP.

The number of filtered candidate values 640 of the first CPP and the number of filtered candidate values 650 of the second CPP may be less than the number of candidate values 610 of the first CPP and the number of candidate values 620 of the second CPP obtained in operation 510. The obtaining of the prediction value of the second target response for the filtered candidate values 640 of the first CPP and the filtered candidate values 640 of the second CPP may have a less number of operations for obtaining the prediction value of the second target response than that of the obtaining of the prediction value of the second target response for the candidate values 610 of the first CPP and the candidate values 620 of the second CPP obtained in operation 510.

Referring to FIG. 5 again, the experimental design method, according to an embodiment, may include operation 550 of outputting an experimental condition set for the CPP by filtering the candidate values of the CPPs based on the condition for the second target response and the prediction value of the second target response. The condition for the second target response is a condition set for the second target response corresponding to the objective of an experiment, and, for example, may include at least one of a condition for maximizing the second target response, a condition for minimizing the second target response, a condition for obtaining the second target response of a value belonging to a certain range, and a condition for obtaining the second target response of a certain value. Based on the condition for the second target response and the prediction value of the second target response, the candidate value of the CPP corresponding to the condition for the second target response may be selected. For example, operation 550 may correspond to the operation of filtering the candidate value of the CPP to be included in an experimental condition set, based on the condition for the target response, described above with reference to FIG. 1.

For example, referring to FIG. 6, the filtered candidate values 640 of the first CPP and the filtered candidate values 650 of the second CPP may be filtered again based on the condition for the second target response and the prediction value of the second target response. Re-filtered candidate values 670 of the first CPP and re-filtered candidate values 680 of the second CPP may be included in the experimental condition set.

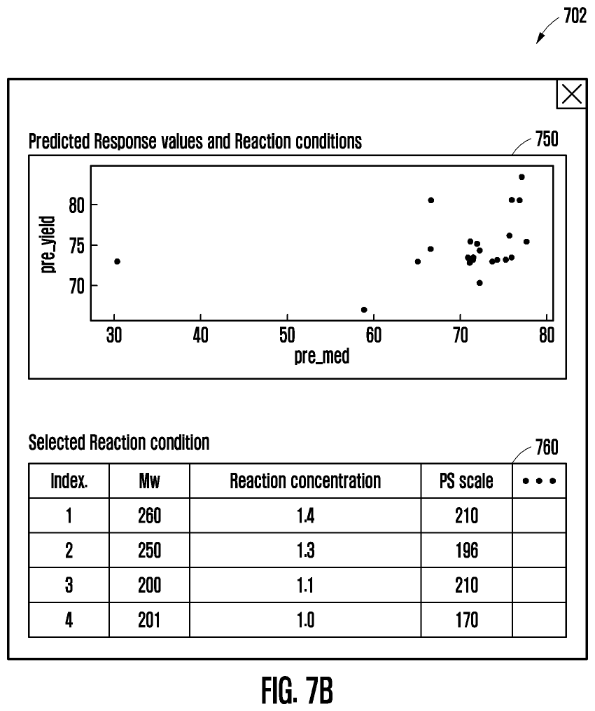

FIGS. 7A and 7B are diagrams each illustrating an interface for providing a method of designing an experiment, according to an embodiment.

The interface for providing a method of designing an experiment may be provided to a user terminal. Hereinafter, the interface for providing a method of designing an experiment may be referred briefly to as the interface. Screen 701 shown in FIG. 7A and screen 702 shown in FIG. 7B may be screens output through the user interface to which the interface is provided. The user terminal may output data upon the execution of the experimental design method through the interface and may receive data input from a user.

Referring to the screen 701 shown in FIG. 7A, the interface for providing a method of designing an experiment may include a target response input window 710 for setting a target response. The user may set the target response by inputting the name of the target response or the identification information of the target response to the target response input window 710. For example, the target response input window 710 may provide a list of target responses to be set and may set an item selected from the list of target responses as the target response. For example, the user may input ‘yield’ to the target response input window 710, and the target response may be set to the yield.

The interface may include a condition input window 720 for the target response to set the condition for the target response. For example, the condition for the target response may be set to a condition for maximizing the target response by an input of selecting a ‘Max’ button 721, the condition for the target response may be set to a condition for minimizing the target response by an input of selecting a ‘Min’ button 722, and the condition for the target response may be set to a condition for obtaining the target response of a certain value by an input of selecting a ‘Value’ button 723. Although not shown in FIG. 7A, if the condition for the target response is set to the condition for obtaining the target response of a certain value, an input window to which the certain value may be input may be displayed, and the certain value may be set. The condition input window 720 for the target response shown in FIG. 7A is just an example of the interface for setting the condition for the target response, and examples are not limited thereto.

The interface may include interfacing objects 730 and 740 for setting a range of a value of each CPP. For example, CPPs may include molecular weights (Mw) and reaction concentration. The interfacing object 730 for setting a range of molecular weight values and the interfacing object 740 for setting a range of reaction concentration values may be provided. Although not shown in FIG. 7A, an interfacing object for setting a range of reaction concentration values may also be provided.

For example, the interfacing object 730 for setting a range of molecular weight values may include a graph having molecular weight values as the horizontal axis and experiment frequency as the vertical axis. The graph of the interfacing object 730 may indicate the molecular weight values included in actual experimental data for obtaining the yield, which is the target response, from molecular weights and the experiment frequency at which the molecular weight values are used for actual experiments. A range 731 of molecular weight values may be set by an input of selecting the molecular weight values in the graph of the interfacing object 730. For example, by an input of selecting two molecular weight values 732 and 733 displayed in the graph of the interfacing object 730, the range 731 of molecular weight values may be determined with the molecular weight value 733, which is a greater value between the two molecular weight values 732 and 733, as the upper limit and the molecular weight value 732, which is a less value between the two molecular weight values 732 and 733, as the lower limit. By providing information on molecular weight values that are used widely in the actual experiments through the graph of the interfacing object 730, the user's setting of the range of molecular weight values may be assisted.

Once an input value for an experimental design is set through the interface, based on the set input value, an experimental condition set of a CPP obtained through the experimental design method may be provided.

Referring to the screen 702 shown in FIG. 7B, a prediction value of the target response for a value of the CPP included in the experimental condition set may be provided through graph 750. Graph 750 may include a graph having the value of the CPP (e.g., reaction concentration) as the horizontal axis and a value of the target response (e.g., yield) as the vertical axis. An experimental condition corresponding to the value of the CPP corresponding to the prediction value of a certain target response may be obtained through graph 750.

Table 760 of the experimental condition set corresponding to graph 750 may be provided. Table 760 may correspond to a table indicating the experimental condition set. Each row of table 760 may indicate a set of values of four CPPs (e.g., molecular weight (Mw), reaction concentration, reaction scale, etc.) included in the experimental condition set.

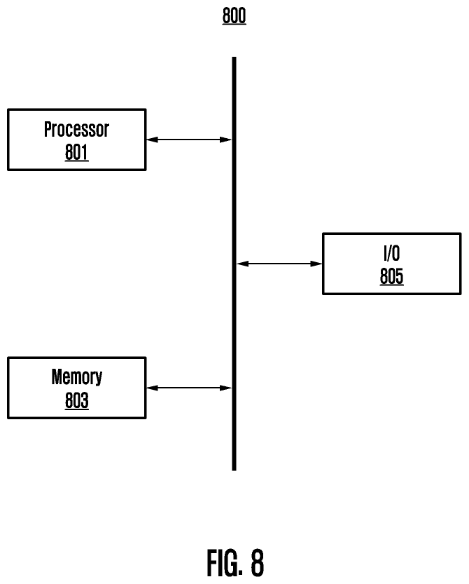

FIG. 8 is a diagram illustrating a configuration of an apparatus according to an embodiment.

Referring to FIG. 8, an apparatus 800 may include a processor 801, a memory 803, and an input/output (I/O) device 805. The apparatus 800 is an apparatus for performing the experimental design method described above with reference to FIGS. 1 to 7 and may include, for example, at least one of a server and a user terminal (e.g., a PC, a smartphone, a tablet, a wearable device, etc.).

The processor 801 may perform at least one operation of the experimental design method described above with reference to FIGS. 1 to 7. For example, the processor 801 may perform at least one of an operation of generating a candidate value of a CPP based on a condition for the CPP corresponding to a target response, an operation of obtaining a prediction value of the target response for the candidate value of the CPP, based on a response prediction model trained to estimate a function of the target response for the CPP, and an operation of outputting an experimental condition set of the CPP based on the prediction value of the target response.

The memory 803 may be a volatile or non-volatile memory and may store data related to the experimental design method described with reference to FIGS. 1 to 7. For example, the memory 803 may store data generated during the process of performing the experimental design method or data necessary for performing the experimental design method. For example, the memory 803 may store the condition for the CPP, a condition for the target response, or weight(s) between layers included in a neural network of the response prediction model.

The apparatus 800 may be connected to an external device (e.g., a server, a user terminal, or a network) through the I/O device 805 to exchange data with the external device. For example, the apparatus 800 may receive an input for setting the condition for the CPP or the condition for the target response from the user through the I/O device 805 and may output the experimental condition set of the CPP.

According to an example, the memory 803 may store a program configured to implement the experimental design method described above with reference to FIGS. 1 to 7. The processor 801 may execute the program stored in the memory 803 and may control the apparatus 800. Code of the program executed by the processor 801 may be stored in the memory 803.

The apparatus 800 according to an embodiment may further include other components not shown in the drawings. For example, the apparatus 800 may further include a communication module. The communication module may provide a function for the apparatus 800 to communicate with other electronic devices or other servers through a network. In addition, for example, the apparatus 800 may further include other components, such as a transceiver, various sensors, or a database.

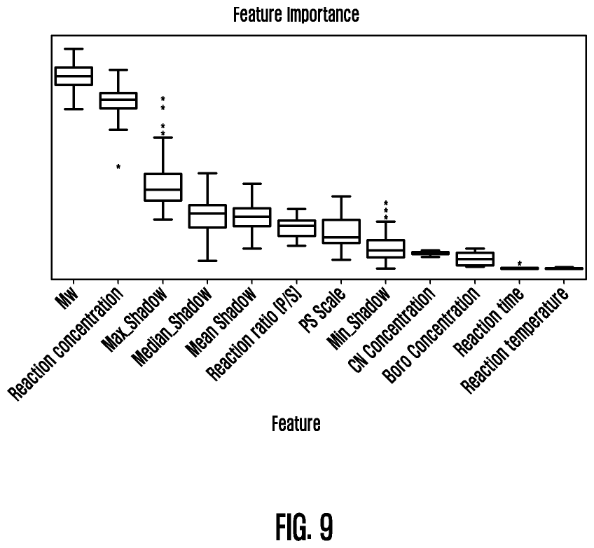

FIG. 9 is a chart illustrating a degree of impact of a process parameter on the determining of a target response, according to an embodiment.

The chart of FIG. 9 may correspond to data output in a multivariate analysis model for evaluating the contribution of process parameters for the target response, according to an embodiment.

Referring to FIG. 9, each of features shown in the horizontal axis or the x-axis may correspond to a process parameter, and the vertical axis or the y-axis may correspond to a value indicating the degree of impact on determining the target response or the feature importance of the target response. Mw and reaction concentration, which are two process parameters having high importance, may be determined as CPPs.

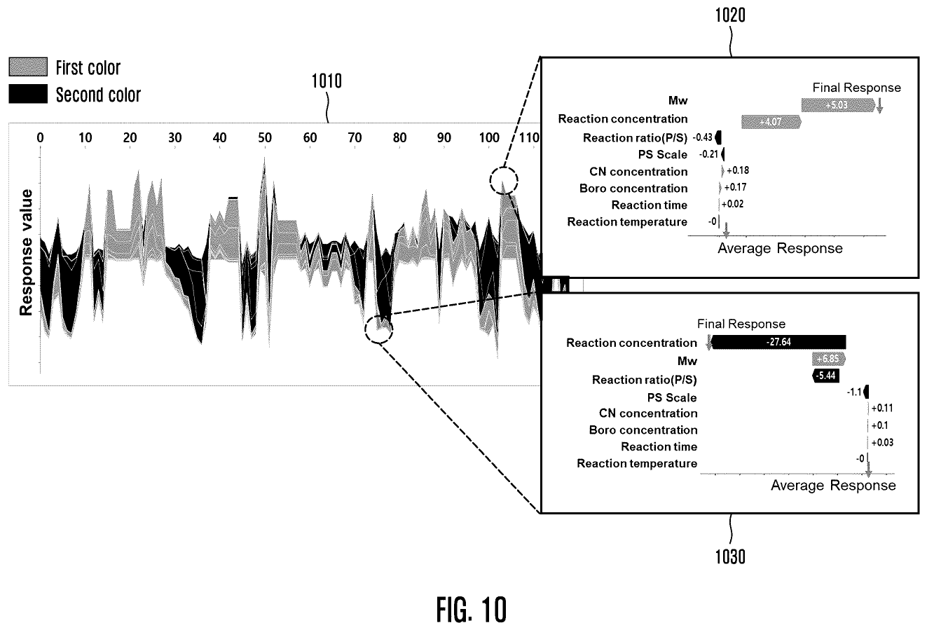

FIG. 10 is a graph illustrating the importance of a process parameter to a target response by experiment, according to an embodiment.

Graph 1010 and charts 1020 and 1030 of FIG. 10 may be data output from an apparatus for an experimental design method, according to an embodiment.

Referring to FIG. 10, each point of graph 1010 refers to a target response value according to a plurality of process parameter values, in which a process parameter affecting an increase of the target response value from an average target response value has a first color, and a process parameter affecting a decrease of the target response value from the average target response value has a second color.

Charts 1020 and 1030 may be charts analyzing process parameters which affect the increasing or decrease from the average target response value in detail. The degree of impact of each process parameter on a change of the target response may be identified quantitatively through charts 1020 and 1030. In charts 1020 and 1030, a process parameter affecting an increase of the target response value from an average target response value has a first color, and a process parameter affecting a decrease of the target response value from the average target response value has a second color.

A result that synthesizes the degree of increasing or decreasing impact of all the process parameters in the average target response value may be estimated as the final target response value. By doing so, a process parameter that causes experimental data to deviate significantly from the average target response value may be identified rapidly.

The units described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor, or any other device capable of responding to and executing instructions in a defined manner. The processing device may run an operating system (OS) and one or more software applications that run on the OS. The processing unit also may access, store, manipulate, process, and generate data in response to execution of the software. For purpose of simplicity, the description of a processing unit is used as singular; however, one skilled in the art will appreciate that a processing unit may include multiple processing elements and multiple types of processing elements. For example, the processing unit may include a plurality of processors, or a single processor and a single controller. In addition, different processing configurations are possible, such as parallel processors.

The software may include a computer program, a piece of code, an instruction, or some combination thereof, to independently or collectively instruct or configure the processing unit to operate as desired. Software and data may be stored in any type of machine, component, physical or virtual equipment, or computer storage medium or device capable of providing instructions or data to or being interpreted by the processing unit. The software also may be distributed over network-coupled computer systems so that the software is stored and executed in a distributed fashion. The software and data may be stored by one or more non-transitory computer-readable recording mediums.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random-access memory (RAM), flash memory, and the like. Examples of program instructions include both machine code, such as produced by a compiler, and files containing higher-level code that may be executed by the computer using an interpreter.

The above-described devices may act as one or more software modules in order to perform the operations of the above-described examples, or vice versa.

As described above, although the examples have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Therefore, other implementations, other examples, and equivalents to the claims are also within the scope of the following claims.

What is claimed is:

1. A method of designing an experiment, the method comprising:
generating a candidate value of a critical process parameter (CPP) based on a condition for the CPP corresponding to a target response;
obtaining a prediction value of the target response for the candidate value of the CPP, based on a response prediction model trained to estimate a function of the target response for the CPP; and
outputting an experimental condition set of the CPP based on the prediction value of the target response.

2. The method of claim 1, further comprising training the response prediction model based on experimental data for measuring the target response for the CPP.

3. The method of claim 1, further comprising determining at least some of process parameters of the CPP, based on a multivariate analysis model for evaluating the contribution of the process parameters for the target response.

4. The method of claim 3, wherein the multivariate analysis model comprises a model for evaluating the contribution of the process parameters for the target response by estimating a Shapley additive explanations (SHAP) value for the target response of the process parameters.

5. The method of claim 3, further comprising determining a training algorithm of the multivariate analysis model based on an evaluation metric of the training algorithm for experimental data for training of the multivariate analysis model; and
training the multivariate analysis model based on the determined training algorithm.

6. The method of claim 5, wherein the training algorithm comprises at least one of random forest, gradient boosted trees, ridge (L2) regression, lasso (L1) regression, light gradient boosting machine (GBM), XGBoost, and decision trees.

7. The method of claim 1, further comprising determining a range of a value of the CPP, based on experimental data for measuring the target response from at least some of the process parameters.

8. The method of claim 7, further comprising setting a range selected by an input of a user within a range of the determined range of the value of the CPP as the condition for the CPP.

9. The method of claim 1, wherein the outputting the experimental condition set of the CPP comprises:
filtering the candidate value of the CPP to be comprised in the experimental condition set, based on a condition for the target response.

10. The method of claim 1, wherein the generating the candidate value of the CPP comprises:
determining a random number satisfying the condition for the CPP as the candidate value of the CPP.

11. The method of claim 1, wherein the generating the candidate value of the CPP comprises:
obtaining the candidate value of the CPP from a language model, based on a prompt corresponding to a condition for the target response and experimental data for measuring the target response for the CPP.

12. The method of claim 11, wherein the generating the candidate value of the CPP from the language model comprises:
obtaining embedding data of the experimental data for measuring the target response for the CPP; and
obtaining the candidate value of the CPP from the language model, based on the prompt corresponding to the condition for the target response and the embedding data of the experimental data.

13. A method of designing an experiment, the method comprising:
generating a candidate value of a critical process parameter (CPP) based on a condition for the CPP;
obtaining a prediction value of a first target response for the candidate value of the CPP;
filtering the candidate value of the CPP based on a condition for the first target response and the prediction value of the first target response;
obtaining a prediction value of a second target response for the filtered candidate value of the CPP; and
outputting an experimental condition set of the CPP by filtering the candidate value of the CPP based on a condition for the second target response and the prediction value of the second target response.

14. The method of claim 13, wherein the obtaining the prediction value of the first target response comprises:
obtaining the prediction value of the first target response for the candidate value of the CPP, based on a response prediction model trained to estimate a function of the first target response for the CPP.

15. The method of claim 13, wherein the obtaining the prediction value of the second target response comprises:
obtaining the prediction value of the second target response for the candidate value of the CPP, based on a response prediction model trained to estimate a function of the second target response for the CPP.

16. A non-transitory computer-readable storage medium storing instructions that, when executed by a processor, cause the processor to perform the method of claim 1.

17. An apparatus comprising a processor configured to
generate a candidate value of a critical process parameter (CPP) based on a condition for the CPP corresponding to a target response,
obtain a prediction value of the target response for the candidate value of the CPP, based on a response prediction model trained to estimate a function of the target response for the CPP, and
output an experimental condition set of the CPP based on the prediction value of the target response.

18. An apparatus comprising a processor configured to
generate a candidate value of a critical process parameter (CPP) based on a condition for the CPP,
obtain a prediction value of a first target response for the candidate value of the CPP,
filter the candidate value of the CPP based on a condition for the first target response and the prediction value of the first target response,
obtain a prediction value of a second target response for the filtered candidate value of the CPP, and
output an experimental condition set of the CPP by filtering the candidate value of the CPP based on a condition for the second target response and the prediction value of the second target response.
